(19) United States
(12) Patent Application Publication
(10) Pub. No.: US 2012/0270276 A1
(43) Pub. Date: Oct. 25, 2012

(54) METHOD FOR PRODUCING SACCHARIDES CONTAINING GLUCOSE AS MAIN COMPONENTS

(75) Inventors: Daisuke Taneda, Ibaraki (JP); Yoshiki Ueno, Ibaraki (JP)

(73) Assignee: JGC Corporation, Tokyo (JP)

(21) Appl. No.: 13/515,817

(22) PCT Filed: Dec. 9, 2010
(86) PCT No.: PCT/JP2010/072153

§ 371 Date: Jun. 14, 2012

(30) Foreign Application Priority Data
Dec. 18, 2009 (JP) 2009-287678

(51) Int. Cl. C12P 19/14

(52) U.S. Cl. 435/99

(57) ABSTRACT

Provided is a method for producing saccharides containing glucose as the main components which can increase the generated amount of saccharides containing glucose as the main components even when an enzymatic saccharification reaction is performed with a small amount of enzyme. A method for producing saccharides containing glucose as the main components is provided which includes mixing biomass containing cellulose and/or hemicellulose and an aqueous enzyme solution, and then performing an enzymatic saccharification reaction with an enzyme contained in the aqueous enzyme solution while maintaining the state where the mixture of the biomass containing cellulose and/or hemicellulose and the aqueous enzyme solution is allowed to stand still in a reaction vessel.

### BACKGROUND OF THE INVENTION

1. Field of the Invention

The present invention relates to a method for producing saccharides containing glucose as the main components employing an enzymatic saccharification reaction of generating saccharides containing glucose as the main components by enzymatic hydrolysis of cellulose or hemicellulose contained in biomass and particularly relates to a method for producing saccharides containing glucose as the main components which can increase the generated amount of glucose even when performing an enzymatic saccharification reaction with a small amount of enzyme.

This application claims the benefit of Japanese Patent Application No. 2009-287678 filed Dec. 18, 2009, which is hereby incorporated by reference herein.

2. Description of the Related Art

At present, techniques for producing bioethanol containing cellulosic biomass as raw materials have been researched and developed in all the countries of the world. The cellulosic biomass refers to trees or grass, agricultural residues, used paper, paper sludge, cotton fibers, and the like and, specifically, construction waste materials or thinnings, rice straw or bagasse (substances that remain after juice is extracted from sugar cane), corn stover, and the like are mentioned.

As a method for producing bioethanol by fermenting saccharide generated from cellulosic biomass, a concentrated sulfuric acid method, a diluted sulfuric acid method, an enzyme method, and the like have been developed. In recent years, particularly the enzyme method among the production methods has drawn attention. The enzyme method is a method for generating ethanol by decomposing cellulose and hemicellulose contained in biomass with enzymes to generate saccharides, and then fermenting the saccharides with fermentative microorganism, such as a yeast.

Since cellulose is a simple polysaccharide in which glucose is dehydrated and polymerized, glucose is generated when cellulose is hydrolyzed (enzymatic hydrolysis) with enzymes.

Since hemicellulose is a complex polysaccharide in which glucose, xylose, mannose, and the like are hydrated and polymerized, glucose, xylose, mannose, and the like are generated when hemicellulose is hydrolyzed (enzymatic hydrolysis) with enzymes.

Then, by adding fermentative microorganism to a solution containing saccharides obtained by such an enzymatic saccharification reaction of cellulose or hemicellulose to ferment the saccharides, ethanol is generated.

Heretofore, in the enzymatic saccharification reaction of cellulose or hemicellulose, a mixed solution (slurry) containing a mixture of biomass containing cellulose and/or hemicellulose and an aqueous solution containing an enzyme (aqueous enzyme solution) is prepared, and the reaction is performed while stirring the slurry in order to accelerate the reaction. Therefore, a large number of study cases on the slurry stirring conditions or a slurry stirring device have been reported. For example, study researches on the influence of the slurry stirring rate, the shape of a stirring blade, the structure of a stirring device, and the like have been reported (e.g., Non-Patent Documents 1 to 4).

As described above, in the researches of the enzymatic saccharification reaction of biomass containing cellulose and/or hemicellulose, researches for optimizing the slurry stirring conditions or the slurry stirring device have been actively performed but researches for performing an enzymatic saccharification reaction without stirring the slurry have not been performed.

This is because the stirring is the basis of reaction operation. For example, when reacting A and B to generate C, performing the reaction while stirring and mixing A and B is the basic common sense in a reaction device. Mentioned as effects obtained by stirring are (1) increasing the contact efficiency of A and B, (2) equalizing the reaction temperature (accelerating heat transfer), (3) equalizing the reaction liquid (slurry) concentration, and the like.

It has also been reported that, in the enzymatic saccharification reaction of biomass containing cellulose and/or hemicellulose, the generated amount of saccharides containing glucose as the main components by the reaction tends to decrease when the amount of the enzyme to be used is small (e.g.,). However, causes of the tendency or measures for improving the tendency have not been clarified.

### RELATED ART

### Non-Patent Document

Non-Patent Document 1: M. Sakata, H. Ooshiima, Y. Harano, and “EFFECTS OF AGITATION ON ENZYMATIC SACCHARIFICATION OF CELLULOSE” Biotechnology Letters, Vol. 7, No. 9, pp. 689-694 (1985)

Non-Patent Document 2: Hanna Ingesson, Guido Zacchi, Bin Yang, Ali R. Esteghlalian, John N. Saddler “The effect of shaking regime on the rate and extent of enzymatic hydrolysis of cellulose” Journal of Biotechnology 88, pp. 177-182 (2001)

Non-Patent Document 3: Henning Jorgensen, Jakob Vibe-Pedersen, Jan Larsen, Claus Felby, “Liquefaction of Lignocellulose at High-Solids Concentrations” Biotechnology and Bioengineering, Vol. 96, No. 5, pp. 862-870, Apr. 1, 2007

Non-Patent Document 4: Tadashi Sakurai, Yasuyuki Takahata, Koji Takahashi, “Stirring operation in enzymatic saccharification of woody biomass” Chemical Engineering, March, 2009, pp 68-72

Non-Patent Document 5: W. Sattler, H. Exterbauer, O. Glatter, W. Steiner, “The Effect of Enzyme Concentration on the Rate of the Hydrolysis of Cellulose” Biotechnology and Bioengineering, Vol. 33, pp. 1221-1234 (1989)

Non-Patent Document 6: Yanpin Lu, Bin Yang, David Gregg, John N. Saddler, Shawn D. Mansfield “Cellulase Adsorption and an Evaluation of Enzyme Recycle During Hydrolysis of Steam-Exploded Softwood Residues” Applied Biochemistry and Biotechnology, Vols. 98-100, 2002

Non-Patent Document 7: Farzaneh Teymouri, Lizbeth Laureano-Perez, Hasan Alizadeh, Bruce E. Dale “Optimization of the ammonia fiber explosion (AFEX) treatment parameters for enzymatic hydrolysis of corn stover” Bioresource Technology 96, pp. 2014-2018, 2005

Non-Patent Document 8: Ming Chen, Liming Xia, Peijian Xue “Enzymatic hydrolysis of corncob and ethanol production from cellulosic hydrolysate” International Biodeterioration & Biodegradation 59 (2007) 85-89

### DISCLOSURE OF THE INVENTION

### Problems to be Solved by the Invention

However, the enzymes to be used in the enzymatic saccharification reaction of cellulose and/or hemicellulose are expensive. Therefore, a method which increases the generated amount of saccharides containing glucose as the main components with a small amount of enzyme by effectively utilizing the enzymes has been desired.

The present invention has been made in view of the above-described circumstances. An objective of the invention is to provide a method for producing saccharides containing glucose as the main components which can increase the generated amount of saccharides containing glucose as the main components even when the enzymatic saccharification reaction is performed with a small amount of enzyme.

### Means for Solving the Subject

A method for producing saccharides containing glucose as the main components, includes mixing biomass containing cellulose and/or hemicellulose and an aqueous enzyme solution, and then performing an enzymatic saccharification reaction of saccharifying the cellulose and/or hemicellulose with an enzyme contained in the enzyme solution while maintaining a state where the mixture of the biomass containing cellulose and/or hemicellulose and the aqueous enzyme solution is allowed to stand still in a reaction vessel.

As the biomass containing cellulose and/or hemicellulose, one which has been pretreated is preferably used.

It is preferable that the enzymatic saccharification reaction proceeds, so that the mixture is phase-separated into solid and liquid phases, which a slurry phase containing the cellulose and/or hemicellulose which are/is undecomposed as the main components and an aqueous glucose solution phase containing an aqueous solution containing glucose generated by the enzymatic saccharification reaction as the main components, and then the enzymatic saccharification reaction is performed while maintaining the state where the mixture is phase-separated into the solid and liquid phases.

It is preferable that the solid and liquid phases are intermittently stirred to temporarily mix the solid and liquid phases, the reactant which becomes uniform is allowed to stand still again, and then the state where the mixture is phase-separated into the solid and liquid phases is maintained.

It is preferable that the time T₁ of stirring the solid and liquid phases and the time T₂ of maintaining the state where the mixture is phase-separated into the solid and liquid phases satisfies the relationship of T₂>T₁.

It is preferable that the reactant is continuously stirred while maintaining the state where the mixture is phase-separated into the solid and liquid phases.

It is preferable that the aqueous solution containing glucose as the main components is extracted, and then the extracted aqueous solution is continuously or intermittently supplied to the slurry phase.

It is preferable that the time t₁ of supplying the aqueous solution containing glucose as the main components to the slurry phase and the time t₂ of maintaining the state where the mixture is phase-separated into the solid and liquid phases satisfies the relationship of t₂>t₁.

It is preferable that the aqueous solution containing glucose as the main components is extracted from the aqueous glucose solution phase while maintaining the state where the mixture is phase-separated into the solid and liquid phases, and then the extracted aqueous solution is continuously supplied to the slurry phase.

### Effect of the Invention

According to the method for producing saccharides containing glucose as the main components of the invention, biomass containing cellulose and/or hemicellulose and an aqueous enzyme solution are mixed in a reaction vessel, and then an enzymatic saccharification reaction is allowed to proceed by controlling the temperature while maintaining the state where the mixture of the biomass containing cellulose and/or hemicellulose and the aqueous enzyme solution is allowed to stand still.

By adopting the reaction system, enzyme degradation due to stirring is suppressed and a state is formed in which particles of cellulose and/or hemicellulose contact with each other in the mixture, so that the contact efficiency of a substrate (cellulose and/or hemicellulose) adhesion enzyme and the cellulose and/or hemicellulose improves, whereby the enzymatic saccharification reaction can be efficiently performed.

Accordingly, the enzyme contained in the aqueous enzyme solution can be effectively utilized. Therefore, even when the enzymatic saccharification reaction is performed with a small amount of enzyme, the generated amount of saccharides containing glucose as the main components can be increased. Moreover, since a stirring device (stirring power) becomes unnecessary, a reduction in the cost of equipment and a reduction in the energy consumption amount can be realized.

### BRIEF DESCRIPTION OF THE DRAWINGS

FIG. 1 is a graph illustrating the results of Example 1 and Comparative Examples 1 and 2 of the method for producing saccharides containing glucose as the main components of the invention.

FIG. 2 is a graph illustrating the results of Example 2 and Comparative Example 3 of the method for producing saccharides containing glucose as the main components of the invention.

FIG. 3 is a graph illustrating the results of Example 3 and Comparative Example 4 of the method for producing saccharides containing glucose as the main components of the invention.

### DESCRIPTION OF THE PREFERRED EMBODIMENTS

Embodiments of a method for producing saccharides containing glucose as the main components are described.

The embodiments specifically describe the invention for understanding the gist of the invention better, and do not limit the invention unless otherwise specified.

The method for producing saccharides containing glucose as the main components of the invention includes mixing biomass containing cellulose and/or hemicellulose and an aqueous enzyme solution, and then performing an enzymatic saccharification reaction of saccharifying the cellulose and/or hemicellulose with the enzyme contained in the aqueous enzyme solution while maintaining the state where the mixture of the biomass containing cellulose and/or hemicellulose and the aqueous enzyme solution is allowed to stand still in a reaction vessel.

Hereinafter, the biomass containing a mixture of cellulose and hemicellulose or the biomass containing cellulose is generically referred to as a cellulose raw material in some cases.

According to the method for producing glucose of the invention, first, the cellulose raw material and an aqueous solution (aqueous enzyme solution) containing an appropriate amount of an enzyme for decomposition of the cellulose raw material are placed in a reaction vessel (enzyme decomposition vessel), and the cellulose raw material and the aqueous enzyme solution are mixed (preparation process).

In the preparation process, the pH of the aqueous enzyme solution is adjusted in such a manner as to achieve pH conditions which are the most optimum for the enzyme to react. Furthermore, the temperature of the aqueous enzyme solution is adjusted in such a manner as to achieve temperature conditions which are the most optimum for the enzyme to react.

In the preparation process, the pH of the mixture of the cellulose raw material and the aqueous enzyme solution is preferably adjusted in such a manner that the enzyme actively functions, and specifically, the pH of the aqueous enzyme solution is preferably adjusted to 4 to 6.

Moreover, in the preparation process, the temperature of the mixture is preferably adjusted in such a manner that the enzyme actively functions, and specifically, the temperature of the aqueous enzyme solution is preferably increased to 50 to 60° C.

The ratio (the addition ratio of the cellulose raw material to the aqueous enzyme solution) of the cellulose raw material to be added to the aqueous enzyme solution is preferably 5 g to 50 g based on 100 mL of the aqueous enzyme solution, i.e., 5 w/v % to 50 w/v % and more preferably 10 g to 40 g based on 100 mL of the aqueous enzyme solution i.e., 10 w/v % to 40 w/v %.

The addition amount of the enzyme contained in the aqueous enzyme solution is preferably 0.1 mg/g to 50 mg/g and more preferably 0.5 mg/g to 30 mg/g per unit cellulose or per weight of cellulose and hemicellulose.

As the enzyme for decomposing cellulose, cellulase is preferably used.

When a large amount of hemicellulose is contained in the cellulose raw material, xylanase and mannanase are preferably added, in addition to cellulase, as the enzyme for decomposing hemicellulose.

Used as the cellulose raw material are (1) one obtained by destroying lignin contained in biomass (trees or grass) and subjecting the biomass to pretreatment, such as partially destroying the crystal structure of cellulose, (2) waste-based raw materials containing cellulose as the main components, such as used paper, corrugated paper, and papermaking sludge, (3) cotton fiber wastes, such as shirts and towels, and the like.

In the pretreatment process, a method for subjecting biomass to alkaline treatment, organic-solvent treatment, diluted sulfuric acid treatment, hot water treatment, or the like is used.

The waste-based raw materials, such as used paper, corrugated paper, and papermaking sludge, or the cotton fiber wastes, such as shirts and towels, do not require the pretreatment in some cases.

The content (remaining amount) of lignin in the cellulose raw material after subjected to the pretreatment is preferably 15% by weight or lower and more preferably 5% by weight or lower.

According to the method for producing saccharides containing glucose as the main components of the invention, by adjusting the content (remaining amount) of lignin in the cellulose raw material after subjected to the pretreatment within the above-mentioned range to thereby maintain the state where the mixture of the cellulose raw material and the aqueous enzyme solution is allowed to stand still, even when the addition amount of the enzyme to cellulose and hemicellulose is small (0.1 mg/g to 50 mg/g per weight of cellulose and hemicelluloses), the enzyme contained in the aqueous enzyme solution is more efficiently utilized, whereby the generated amount of saccharides containing glucose as the main components can be increased even when the enzymatic saccharification reaction of saccharifying cellulose and/or hemicellulose with a small amount of enzyme, in addition the energy saving effect of saving a stirring device or stirring power can be obtained.

However, when the content of lignin in the cellulose raw material exceeds 15% by weight, it does not result in that the effects of the invention are not demonstrated at all. More specifically, even when the content of lignin in the cellulose raw material exceeds 15% by weight, a tendency is observed in which the enzymatic saccharification reaction becomes slower than in the case where the content of lignin is 15% by weight or lower, so that it becomes difficult to increase the generated amount of saccharides containing glucose as the main components, but the enzymatic saccharification reaction sufficiently proceeds. Accordingly, in the invention, even when the content of lignin exceeds 15% by weight, the energy saving effects of saving a stirring device or stirring power, which has been used for performing the enzymatic saccharification reaction heretofore, can be obtained.

Subsequently, by adjusting the temperature of the mixture while maintaining the state where the mixture of the cellulose raw material and the aqueous enzyme solution is allowed to stand still, the enzymatic hydrolysis of the cellulose raw material contained in the aqueous enzyme solution is performed for saccharification of the cellulose raw material to thereby generate saccharides containing glucose as the main components and containing mannose, xylose, and the like (enzymatic saccharification reaction process).

In the invention, the “state of being allowed to stand still” means the state where the mixture of the cellulose raw material and the aqueous enzyme solution is left as it is without stirring.

In the enzymatic saccharification reaction, the temperature of the mixture is preferably adjusted in such a manner that the enzyme actively functions, and, specifically, the temperature is preferably held at 50 to 60° C.

The enzymatic saccharification reaction is performed until the saccharification of the cellulose raw material with enzymes sufficiently proceeds, and then the reaction does not proceed any more. For example, the enzymatic hydrolysis of the cellulose raw material is performed at 50 to 60° C. for about 2 days to 60 days.

Herein, reasons why an efficient enzymatic saccharification reaction can proceed with a small amount of enzyme by performing the enzymatic saccharification reaction of the mixture of the cellulose raw material and the aqueous enzyme solution while maintaining the state where the mixture is allowed to stand still in the enzymatic saccharification reaction process are considered as follows.

When the mixture of the cellulose raw material and the aqueous enzyme solution is not stirred, the enzyme degradation due to stirring is suppressed and the cellulose raw materials contact with each other in the mixture, so that the contact efficiency of the enzyme adhering to the substrate and the cellulose raw material increases, whereby the enzymatic saccharification reaction can be efficiently performed.

Accordingly, the enzyme contained in the aqueous enzyme solution can be effectively utilized. Therefore, even when the enzymatic saccharification reaction is performed with a small amount of enzyme, the generated amount of saccharides containing glucose as the main components can be increased.

Moreover, by maintaining the state where the mixture is allowed to stand still without stirring, the equalization rate of the temperature or the concentration in the reaction vessel becomes low. However, the enzymatic saccharification reaction rate is lower than the heat transfer rate or the diffusion rate, and therefore negative influence due to not stirring is less.

Furthermore, when heat transfer or diffusion causes problems, there are a method for intermittently stirring in a short time, a method for stirring at a low number of rotations while maintaining the state where the mixture is phase-separated into the solid and liquid phases, and the like.

The enzymatic hydrolysis of the cellulose raw material proceeds, so that the mixture of the cellulose raw material and the aqueous enzyme solution is phase-separated into solid and liquid phases of an aqueous glucose solution phase (upper phase) containing glucose generated by the enzymatic hydrolysis of the cellulose raw material as the main components and a slurry phase (lower phase) containing an undecomposed cellulose raw material as the main components, and then the above-described enzymatic saccharification reaction is preferably performed while maintaining the state where the mixture is phase-separated into the aqueous glucose solution phase and the slurry phase.

When the enzymatic hydrolysis of the cellulose raw material is performed, glucose of the decomposition product serves as a reaction inhibitor of the enzymatic hydrolysis. Therefore, when the glucose concentration in the slurry phase in which the enzymatic hydrolysis is performed becomes high, it is preferable to temporarily or intermittently stir the solid and liquid phases containing the aqueous glucose solution phase and the slurry phase in order to lower the glucose concentration.

It is also preferable that, after simultaneously intermittently stirring the aqueous glucose solution phase and the slurry phase to temporarily mix the aqueous glucose solution phase and the slurry phase, the reactant which has become uniform is allowed to stand still again, and then the enzymatic saccharification reaction of the cellulose raw material is performed while maintaining the state where the mixture is phase-separated into the aqueous glucose solution phase and the slurry phase.

As described above, when the enzymatic hydrolysis of the cellulose raw material is performed, glucose of the decomposition product serves as a reaction inhibitor of the enzymatic hydrolysis. Therefore, when the state where the solid-liquid phases are allowed to stand still is continuously maintained from beginning to end, glucose generated by the enzymatic hydrolysis of the cellulose raw material stays around the cellulose raw material contained in the slurry phase, which locally increases the glucose concentration. As a result, the enzymatic hydrolysis reaction does not proceed in some cases. Then, by intermittently stirring the aqueous glucose solution phase and the slurry phase to temporarily mix the solid and liquid phases to relatively reduce the glucose concentration in the slurry phase, the inhibition of the enzymatic hydrolysis by glucose can be suppressed.

When the time of stirring the aqueous glucose solution phase and the slurry phase is defined as T₁ and the time of maintaining the state where the mixture is phase-separated into the aqueous glucose solution phase and the slurry phase is defined as T₂, it is preferable to satisfy the relationship of T₂>T₁.

In the invention, in order to maintain the state where the particles of the cellulose raw material contact with each other, it is necessary to perform the enzymatic hydrolysis of the cellulose raw material while maintaining the state where the slurry phase containing the cellulose raw material as the main components is allowed to stand still as described above. Therefore, the time (T₁) of stirring the aqueous glucose solution phase and the slurry phase may be short. More specifically, the time (T₁) of stirring the aqueous glucose solution phase and the slurry phase may be adjusted in such a manner that the solid and liquid phases are temporarily sufficiently mixed. Therefore, it is preferable to sufficiently lengthen the time T₂ of maintaining the state where the mixture is phase-separated into the aqueous glucose solution phase and the slurry phase relative to the time T₁ of stirring the aqueous glucose solution phase and the slurry phase tp.

The solid and liquid phases may be continuously stirred in the state of the low number of rotations at which the state where the mixture is phase-separated into the aqueous glucose solution phase and the slurry phase can be maintained.

By continuously stirring the solid and liquid phases while maintaining the state where the mixture is phase-separated into the aqueous glucose solution phase and the slurry phase, glucose generated in the slurry phase can be gradually moved to the aqueous glucose solution phase at the interface of the solid and liquid phases, so that the glucose concentration in the slurry phase can be reduced. Therefore, the enzymatic hydrolysis reaction can be allowed to proceed without a hitch.

As a method for reducing the concentration of the glucose generated in the slurry phase, the glucose solution generated in the aqueous glucose solution phase in the reaction vessel may be continuously or intermittently supplied to the slurry phase with a pump, in addition to the above-described method for intermittently stirring the solid and liquid phases or stirring the solid and liquid phases at a low number of rotations.

By supplying the aqueous glucose solution to the slurry phase, the glucose concentration which becomes locally high in the slurry phase can be reduced similarly as in the above-described stirring operation.

When the aqueous glucose solution is continuously supplied to the slurry phase, it is preferable to supply the aqueous glucose solution at a flow rate at which the state where the mixture is phase-separated into two phases of the aqueous glucose solution phase and the slurry phase can be maintained.

When the time of supplying the aqueous solution containing glucose as the main components to the slurry phase is defined as t₁ and the time of maintaining the state where the mixture is phase-separated into the aqueous glucose solution phase and the slurry phase is defined as t₂, it is preferable to satisfy the relationship of t₂>t₁.

In the invention, in order to maintain the state where the particles of the cellulose raw material contact with each other, it is necessary to perform the enzymatic hydrolysis of the cellulose raw material while maintaining the state where the slurry phase containing the cellulose raw material as the main components is allowed to stand still as described above. Therefore, the time (t₁) of supplying the aqueous solution containing glucose as the main components to the slurry phase may be short. More specifically, the time (t₁) of supplying the aqueous solution containing glucose as the main components to the slurry phase may be adjusted in such a manner that the aqueous solution containing glucose as the main components is temporarily sufficiently supplied to the slurry phase. Therefore, it is preferable to sufficiently lengthen the time t₂ of maintaining the state where the mixture is phase-separated into the aqueous glucose solution phase and the slurry phase relative to the time t₁ of supplying the aqueous solution containing glucose as the main components.

Moreover, the aqueous solution containing glucose as the main components may be extracted from the aqueous glucose solution phase while phase-separating the mixture into the aqueous glucose solution phase and the slurry phase, and may be continuously supplied to the slurry phase.

By extracting the aqueous solution containing glucose as the main components from the aqueous glucose solution phase while maintaining the state where the mixture is phase-separated into the aqueous glucose solution phase and the slurry phase, and then continuously supplying the aqueous solution to the slurry phase, the glucose concentration which becomes locally high in the slurry phase can be lowered. Therefore, the enzymatic hydrolysis reaction can be allowed to proceed without a hitch.

According to the method for producing saccharides containing glucose as the main components of the invention, the enzymatic hydrolysis of the cellulose raw material is performed with the enzyme contained in the aqueous enzyme solution while maintaining the state where the mixture of the cellulose raw material and the aqueous enzyme solution is allowed to stand still. Therefore, the state where particles of the cellulose raw material contact with each other in the mixture is maintained to increase the contact efficiency of the enzyme adhering to the substrate and the cellulose raw material, whereby the enzymatic saccharification reaction can be efficiently performed. Accordingly, the enzyme contained in the aqueous enzyme solution can be effectively utilized. Therefore, even when the enzymatic saccharification reaction is performed with a small amount of enzyme, the generated amount of saccharides containing glucose as the main components can be increased. Moreover, since a stirring device becomes unnecessary, a reduction in the cost of equipment and a reduction in the energy consumption amount can be realized.

### EXAMPLES

Hereinafter, the invention is more specifically described according to Examples and Comparative Examples but is not limited to the following examples.

### Example 1

In a reaction vessel, 100 mL of a 50 mM acetic acid buffer solution (pH 5) containing 0.5 mL of a cellulase solution was placed, and then 10 g of a filter paper was immersed as cellulose in the aqueous enzyme solution (Cellulase solution concentration: 0.5% by volume) (Addition amount of cellulase of 5 mg solution/g-Filter paper).

By adjusting the temperature of the mixture to 50° C. while maintaining the state where the mixture was allowed to stand still, the enzymatic hydrolysis of the filter paper was performed with the cellulase contained in the aqueous enzyme solution.

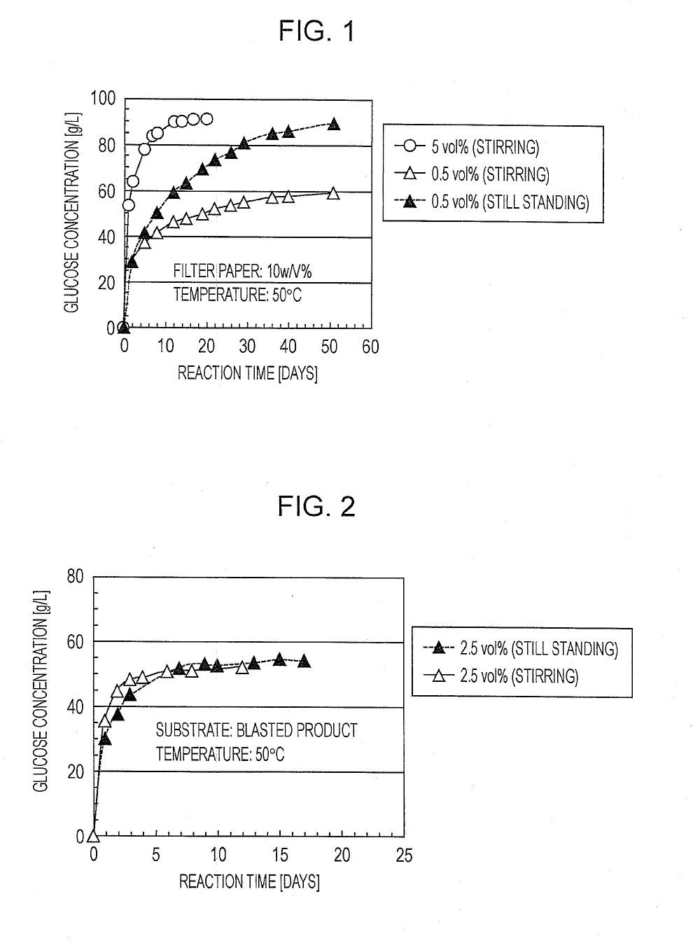

The results are shown in FIG. 1.

### Comparative Example 1

In a reaction vessel, 100 mL of a 50 mM acetic acid buffer solution (pH 5) containing 0.5 mL of a cellulase solution was placed, and then 10 g of a filter paper was immersed as cellulose in the aqueous enzyme solution (Cellulase solution concentration: 0.5% by volume) (Addition amount of cellulase of 5 mg solution/g-Filter paper).

By adjusting the temperature of the mixture to 50° C. while stirring the mixture, the enzymatic hydrolysis of the filter paper was performed with the cellulase contained in the aqueous enzyme solution.

The results are shown in FIG. 1.

### Comparative Example 2

In a reaction vessel, 100 mL of a 50 mM acetic acid buffer solution (pH 5) containing 5 mL of a cellulase solution was placed, and then 10 g of a filter paper was immersed as cellulose in the aqueous enzyme solution (Cellulase solution concentration: 5% by volume) (Addition amount of cellulase of 50 mg solution/g-Filter paper).

By adjusting the temperature of the mixture to 50° C. while stirring the mixture, the enzymatic hydrolysis of the filter paper was performed with the cellulase contained in the aqueous enzyme solution. The results are shown in FIG. 1.

It was confirmed from the results of FIG. 1 that, in Example 1 in which the mixture was allowed to stand still, only 0.5 mL of the cellulase solution was added but glucose can be generated by setting the reaction time to 40 days or longer to the same level as in Comparative Example 2 in which 5 mL of the cellulase solution was added, which is 10 times the addition amount of the cellulase solution in Example 1.

In contrast, in Comparative Example 1 in which the mixture was stirred, the tendency in which the reaction was saturated within about 30 days after the reaction started was observed and also the generated amount of glucose was only half of that of Example 1.

### Example 2

In a reaction vessel, 100 mL of a 50 mM acetic acid buffer solution (pH 5) containing 2.5 mL of a cellulase solution was placed, and then 20 g of wood subjected to blasting treatment was immersed as biomass containing cellulose in the aqueous enzyme solution (Cellulase solution concentration: 2.5% by volume) (Addition amount of cellulase of 12.5 mg solution/g-Blasted product).

By adjusting the temperature of the mixture to 50° C. while maintaining the state where the mixture was allowed to stand still, the enzymatic hydrolysis of the wood subjected to blasting treatment was performed with the cellulase contained in the aqueous enzyme solution. The results are shown in FIG. 2.

### Comparative Example 3

In a reaction vessel, 100 mL of a 50 mM acetic acid buffer solution (pH 5) containing 2.5 mL of a cellulase solution was placed, and then 20 g of wood subjected to blasting treatment was immersed as biomass containing cellulose in the aqueous enzyme solution (Cellulase solution concentration: 2.5% by volume) (Addition amount of cellulase of 12.5 mg solution/g-Blasted product).

By adjusting the temperature of the mixture to 50° C. while stirring the mixture, the enzymatic hydrolysis of the wood subjected to blasting treatment was performed with the cellulase contained in the aqueous enzyme solution. The results are shown in FIG. 2.

From the results of FIG. 2, the glucose concentration in Example 2 in which the mixture was allowed to stand still was hardly different from that of Comparative Example 3 in which the mixture was stirred. More specifically, it was confirmed that same amount of glucose can be produced without stirring from the raw material subjected to blasting treatment with a high lignin content (Lignin content: 50% by weight).

### Example 3

In a reaction vessel, 100 mL of a 50 mM acetic acid buffer solution (pH 5) containing 0.5 mL of a cellulase solution was placed, and then 10 g of paper pulp was immersed as cellulose in the aqueous enzyme solution (Cellulase solution concentration: 0.5% by volume) (Addition amount of cellulase of 5 mg solution/g-Paper pulp).

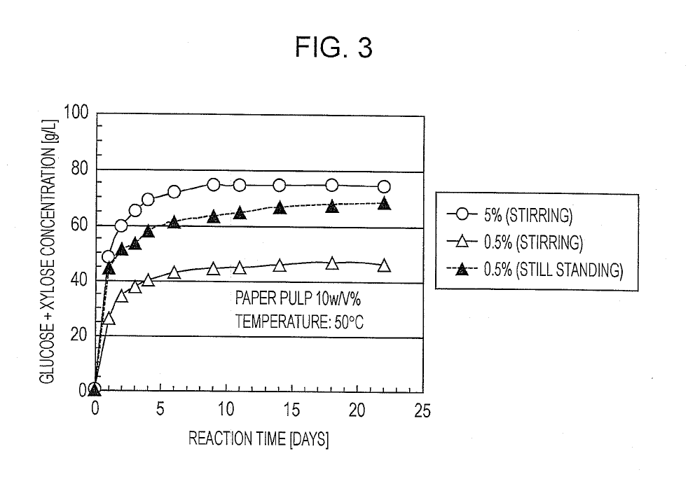

By adjusting the temperature of the mixture to 50° C. while maintaining the state where the mixture was allowed to stand still, the enzymatic hydrolysis of the paper pulp was performed with the cellulase contained in the aqueous enzyme solution. The results are shown in FIG. 3.

### Comparative Example 4

In a reaction vessel, 100 mL of a 50 mM acetic acid buffer solution (pH 5) containing 0.5 mL of a cellulase solution was placed, and then 10 g of paper pulp was immersed as cellulose in the aqueous enzyme solution (Cellulase solution concentration: 0.5% by volume) (Addition amount of cellulase of 5 mg solution/g-Paper pulp).

By adjusting the temperature of the mixture to 50° C. while stirring the mixture, the enzymatic hydrolysis of the paper pulp was performed with the cellulase contained in the aqueous enzyme solution.

The results are shown in FIG. 3.

### Comparative Example 5

In a reaction vessel, 100 mL of a 50 mM acetic acid buffer solution (pH 5) containing 5 mL of a cellulase solution was placed, and then 10 g of paper pulp was immersed as cellulose in the aqueous enzyme solution (Cellulase concentration: 5% by volume) (Addition amount of cellulase of 50 mg/g-Paper pulp).

By adjusting the temperature of the mixture to 50° C. while stirring the mixture, the enzymatic hydrolysis of the paper pulp was performed with the cellulase contained in the aqueous enzyme solution. The results are shown in FIG. 3.

It was confirmed from the results of FIG. 3 that, in Example 3 in which the mixture was allowed to stand still, only 0.5 mL of the cellulase solution was added but a larger amount of glucose can be generated by setting the reaction time to 2 days or longer than the generated amount of glucose in Comparative Example 4 in which the equivalent amount of the cellulase solution was added and, by setting the reaction time to 20 days or longer, the generated amount of glucose which is almost the same as that of Comparative Example 5 in which 5 mL of the cellulase solution was added, which is 10 times the amount of the cellulase solution in Example 3, can be achieved.

### INDUSTRIAL APPLICABILITY

According to the production method of the present invention, the enzyme contained in the aqueous enzyme solution can be effectively utilized. Therefore, even when the enzymatic saccharification reaction is performed with a small amount of enzyme, the generated amount of saccharides containing glucose as the main components can be increased. Moreover, since a stirring device (stirring power) becomes unnecessary, a reduction in the cost of equipment and a reduction in the energy consumption amount can be realized.

1. A method for producing saccharides containing glucose as the main components, comprising:
mixing biomass containing cellulose and/or hemicellulose and an aqueous enzyme solution, and
performing an enzymatic saccharification reaction of saccharifying the cellulose and/or hemicellulose with an enzyme contained in the enzyme solution while maintaining a state where the mixture of the biomass containing cellulose and/or hemicellulose and the aqueous enzyme solution is allowed to stand still in a reaction vessel.

2. The method for producing saccharides containing glucose as the main components according to claim 1, wherein the biomass containing cellulose and/or hemicelluloses is pretreated.

3. The method for producing saccharides containing glucose as the main components according to claim 1, wherein the enzymatic saccharification reaction proceeds, so that the mixture is phase-separated into solid and liquid phases of an aqueous glucose solution phase containing an aqueous solution containing glucose generated by the enzymatic saccharification reaction as the main components and a slurry phase containing the cellulose and/or hemicellulose which are/is undecomposed as the main components, and then the enzymatic saccharification reaction is performed while maintaining the state where the mixture is phase-separated into the solid and liquid phases.

4. The method for producing saccharides containing glucose as the main components according to claim 3, wherein the solid and liquid phases are intermittently stirred to temporarily mix the solid and liquid phases, the reactant which becomes uniform is allowed to stand still again, and then the state where the mixture is phase-separated into the solid and liquid phases is maintained.

5. The method for producing saccharides containing glucose as the main components according to claim 4, wherein the time T₁ of stirring the solid and liquid phases and the time T₂ of maintaining the state where the mixture is phase-separated into the solid and liquid phases satisfies the relationship of T₂>T₁.

6. The method for producing saccharides containing glucose as the main components according to claim 3, wherein the reactant is continuously stirred while maintaining the state where the mixture is phase-separated into the solid and liquid phases.

7. The method for producing saccharides containing glucose as the main components according to claim 3, wherein the aqueous solution containing glucose as the main components is extracted, and then the extracted aqueous solution is continuously or intermittently supplied to the slurry phase.

8. The method for producing saccharides containing glucose as the main components according to claim 7, wherein the time t₁ of supplying the aqueous solution containing glucose as the main components to the slurry phase and the time t₂ of maintaining the state where the mixture is phase-separated into the solid and liquid phases satisfies the relationship of t₂>t₁.

9. The method for producing saccharides containing glucose as the main components according to claim 3, wherein the aqueous solution containing glucose as the main components is extracted from the aqueous glucose solution phase while maintaining the state where the mixture is phase-separated into the solid and liquid phases, and then the extracted aqueous solution is continuously supplied to the slurry phase.
